(19) United States
(12) Patent Application Publication
(10) Pub. No.: US 2025/0022560 A1
(43) Pub. Date: Jan. 16, 2025

(54) INDIVIDUALIZED MANAGEMENT SYSTEM FOR TREATING DISEASE, INCLUDING GI DISEASES, AND METHODS OF USE THEREOF

(71) Applicant: AYBLE HEALTH, INC., Boston, MA (US)

(72) Inventors: Samuel N. JACTEL, Boston, MA (US); Joseph M. OLSON, Boston, MA (US); Stephen E. LUPE, Boston, MA (US); Tiffany N. TAFT, Boston, MA (US); Jennifer H. FISHER, Boston, MA (US)

(73) Assignee: AYBLE HEALTH, INC., Boston, MA (US)

(21) Appl. No.: 18/349,700

(22) Filed: Jul. 10, 2023

(51) Int. Cl. G16H 20/00; G16H 10/60; G16H 50/20

(52) U.S. Cl. CPC G16H 20/00; G16H 10/60; G16H 50/20

(57) ABSTRACT

The invention is generally related to methods and systems to manage Inflammatory Bowel Disease, Irritable Bowel Syndrome and other immune-mediated inflammatory disorders (IMIDs in a subject in need thereof. One embodiment of the present invention contemplates a system and method useful for managing and ameliorating IBD and IBS symptoms encompassing the iterative development of personalized treatment modalities including a diet modality, a psychological behavior module, a stress-reduction modality, a modality related to activity and exercise and the like to aid the participant to reduce or eliminate most IMID symptoms and increase her sense of well-being.

### TECHNICAL FIELD OF THE INVENTION

The methods and systems of the present invention are generally directed to the management of immune-mediated inflammatory disorders (IMIDs), such as Inflammatory Bowel Disease and the like. The present invention contemplates a system allowing for input, collection, and output of data as well as a means for data analysis which provides for the generation of reiterative personalized modalities to allow a participant to beneficially manage an IMID. The personalized program of the present invention contemplates the individual's participation in one or more modalities, such as but not limited to cognitive behavior therapy (CBT), stress reduction techniques, sleep hygiene methods, diet and physical exercise. The data generated by the individual's participation is further collected and analyzed by the system with the resulting modification of the program to optimize the individual's ability to positively affect symptom management and reduce exacerbation of disease symptoms.

### BACKGROUND OF THE INVENTION

### Inflammatory Bowel Diseases:

The inflammatory bowel diseases [IBD], which include Crohn's disease and ulcerative colitis (UC), are chronic relapsing and remitting diseases that cause inflammation of the gastrointestinal tract.¹ Common symptoms include mild presentation such as cramping and diarrhea, whereas more severe cases may include rectal abscess, fistulas and ulcers around the anus. Nor is the pathology of IBD limited to cramping, diarrhea, and the like. It is also recognized that IBD patients are at an increased risk of developing colorectal cancer, primarily as a result of the intestinal inflammation.¹⁴ Treatment of IBD involves a multidisciplinary approach involving both medical therapies, mental health psychotherapy, lifestyle changes, and surgical intervention. Recent advances in drug treatments, including the advent of new biologics, has led to advances in the management of IBD. However, these therapies are expensive and, even before the introduction of newer therapeutic agents, treatment cost in the United States from 2003 to 2004 was estimated to exceed $6.3 billion dollars.¹⁵ Even with the introduction of new and expensive treatment modalities, including anti-tumor necrosis factor, Janus kinase (JAK) inhibitors, immune cell modulators, stem cells, lymphocyte sequestration agents, anti-sense drugs, granulocyte cell stimulating factors, and mucosal barrier enhancers, it is not clear that any of these advances will substantially reduce the requirement for surgery to manage IBD.¹⁶

Since the earliest descriptions of UC and Crohn's disease, inflammatory bowel diseases have grown to be among the most difficult to characterize, difficult to treat, and costly chronic gastrointestinal diseases in the developed world.² Crohn's disease is named after Dr. Burrill B. Crohn, who first described the disease in 1932 along with his colleagues, Dr. Leon Ginzburg and Dr. Gordon D. Oppenheimer. IBD affects an estimated 3 million Americans, with men and women equally likely to be affected by Crohn's disease.³ Although the disease can occur at any age, Crohn's disease is most often diagnosed in adolescents and adults between the ages of 20 and 30, with studies have shown that between 1.5 percent and 28 percent of people with IBD have a first-degree relative, such as a parent, child, or sibling, who also has one of the diseases.⁴ Crohn's disease can affect people from all ethnic backgrounds, although it is more common in Caucasians and recently rates of Crohn's disease have increased among Hispanics and Asians.⁵

Differences amongst the Inflammatory Bowel Diseases can be seen, amongst other factors, based on location of GI tract affected. For example, Crohn's Disease can affect any part of the GI tract from the mouth to the anus, but most commonly affects the end of the small bowel (ileum) and the beginning of the colon. Crohn's affects the entire thickness of the bowel wall, but the resulting inflammation can leave normal areas in between patches of diseased intestine.⁶

The most common type of Crohn's Disease is ileocolitis which affects the terminal ilium, or end of the small intestine, and is characterized by diarrhea, cramping and pain in the middle or lower right part of the abdomen and may be accompanied by significant weight loss. Illeitis is a type of Crohn's that affects only the ileum but exhibits symptoms similar to ileocolitis. Severe cases may include the presence of fistulas or inflammatory abscesses in the right lower quadrant of the abdomen. A third type, known as gastroduodenal Crohn's Disease, affects the stomach and the beginning of the small intestine (duodenum) and is characterized by symptoms including nausea, vomiting, loss of appetite and weight loss. In contrast, jejunoileitis is characterized by patchy areas of inflammation in the upper half of the small intestine (jejunum) and may include mild to intense abdominal pain and cramps following meals and diarrhea and fistulas may form in severe cases or after prolonged periods of inflammation. Crohn's (Granulomatous) Colitis affect only the colon, or large intestine. Common symptoms include diarrhea, rectal bleeding abscess, fistulas and ulcers around the anus. Skin lesions and joint pains are more common in this form of Crohn's than in others.⁷

While the exact etiology of IBD is unknown, the syndrome is characterized by chronic inflammation of the gastrointestinal tract in genetically susceptible individuals exposed to risk factors, including evidence that gut microbiota may play a role.⁸ It is known that the immune system of the gastrointestinal tract plays a major role in providing an appropriate response to harmful pathogens, while inducing an immune tolerance to harmless food materials and commensal flora. That this immune hemostasis is disturbed in patients with IBD is clear and, during the past several decades, epidemiological studies have provided support for the hypothesis that the increase in the incidence of IBD in adults and children may be linked to changes in lifestyle and nutritional habits.⁹ Genetic predisposition plays a key role in vulnerability to Crohn's disease and, to a lesser degree, to ulcerative colitis. These genetic influences account in large part for higher incidences in certain ethnic groups and families.¹⁰ The number of genetic loci associated with different frequencies of IBD is approaching 200, yet in combination they seem to account for less than 15% of variance in Crohn's disease and only 7.5% of variance in ulcerative colitis.¹¹

Additional support indicating the significant role environmental factors play in the development of IBD is illustrated by the disease occurrence pattern evidenced in siblings and in monozygotic twins, the rapidly increasing incidence of IBD over time, and the seismic shifts in incidence among populations of varying ethnicities as they migrate to different geographic areas. Novel technologies have provided an opportunity to scan throughout the genome for finding susceptibility loci associated with IBD. Identification of these variants have elucidated pathways involved in the IBD pathogenesis with the hope for providing better treatments. Recently 163 loci for IBD have been identified, some of which are implicated in other inflammatory diseases; of the 163 loci, 30 loci are specific to CD, 23 loci to UC, and 110 loci are in common.¹²

Most environmental risk factors seem to affect both Crohn's disease and ulcerative colitis in a similar manner, but there are some interesting differences. For example, smoking appears to be protective against ulcerative colitis but worsens the symptoms associated with Crohn's disease, while there is some evidence that appendectomies may be protective against ulcerative colitis but worsen Crohn's disease.¹³

### Irritable Bowel Syndrome:

Similar to IBD, Irritable bowel syndrome (IBS) is a chronic disorder of the gastrointestinal tract, characterized by abdominal pain and alterations in bowel habits 16. Diagnosis is made by a gastroenterologist according to a symptom-based classification system, the Rome Criteria, and patients are categorized according to the predominant bowel habits they experience: IBS-C(constipation), IBS-D (diarrhea), IBS-M (mixed bowel characteristics) or IBS-U (unsubtyped). Quality of life of IBS patients is significantly impacted by symptoms; 40-60% of patients not only experience gastrointestinal distress, but also experience comorbid psychological disorders such as anxiety and depression¹⁷. Other somatic comorbidities include pain syndromes, overactive bladder and migraine.¹⁸

IBS is the most frequently diagnosed chronic gastrointestinal disease. Prevalence rates in North America have been reported to be between 12-15%, but these prevalence numbers may under-report the burden of IBS because of the significant overlap seen in clinical symptoms for this and other GI conditions, as well as the stigmatization of the diagnosis. For instance, there is a reported 40-50% of IBD patients with concomitant IBS. Pooled global prevalence of IBS is estimated to be over 11% in 2016 and growing, particularly in Asian countries. The most important single risk factors for IBS are female sex, younger age and preceding gastrointestinal infections¹⁶⁻¹⁸.

The financial burden these patients generate on the US healthcare system is significant. Each IBS patient on average generates upwards of $18,000 in direct medical spend each year in the United States and the IBS population contributes significantly to the $140 billion/year in direct spend attributed to gastrointestinal disorders.

As with IBD, the etiology of IBS is unclear and likely multifactorial: gastrointestinal dysmotility, inflammation, visceral hypersensitivity, genetic predisposition, GI infections, and altered intestinal microbiota have all been proposed as mechanisms that contribute to symptomatology. Approaches to treatment are similarly diverse, and can include pharmacologic intervention (e.g., antispasmodics, selective serotonin reuptake inhibitors, opioid agonists, antibiotics, bile salt sequestrants), over-the-counter medication (e.g., peppermint oil, probiotics) and behavioral health management (e.g., dietary alteration, gut-directed psychological therapy). There is no cure and no models exist to predict what combination of therapeutic approaches have the highest likelihood of controlling an individual patients' symptoms, but it is generally accepted that multiple therapeutic tools working in tandem are more effective than one alone¹⁶⁻¹⁸.

Food ingestion is one of the most commonly reported factors that results in the exacerbation of symptoms among patients with IBS and IBD; dietary modifications have the potential to generate significant improvement in patients' quality of life and symptom severity. However, until recently, food-related symptoms had received little attention from the literature, leaving patients to find their own way through the plethora of usually non-validated and untested diagnostic tests and dietary regimens, which could result in clinically relevant nutritional deficits. Although patients with IBS and IBD readily incriminate specific food items as those that are especially likely to precipitate symptoms, only 11-27% of those trigger foods are correctly identified when confirmed in formal, blinded food challenge studies.¹⁸

Mental distress is another commonly reported factor that results in the exacerbation of symptoms among patients with IBS and IBD. Psycho-gastroenterological interventions for IBS and IBD (cognitive behavioral therapy, acceptance and commitment therapy, hypnotherapy, resilience therapy and other methods) have shown to improve outcomes, patient quality of life (HRQoL) and long-term disease management. In addition to the impact these interventions can have on GI symptoms, they're also critical in managing the prevalence of anxiety and mood disorders that are comorbid in 30-50% of these patients (up to 90% in treatment-seeking patients). Finally, recent data indicate that psychological distress may be causally associated with both onset and subsequent IBD and IBS activity (https://pubmed.ncbi.nlm.nih.gov/30452919/). Typical gastropsychology programs require patients to complete specific exercises in a specific cadence, with no room for modification, rather than a personalized/curated experience combining multiple therapeutic tools. Adherence rates during study periods have been reported to be high (>75%), but suffer from significant dropout and follow-up rate decline between 25-62%.

### Other Immune Mediated Inflammatory Disorders (IMID):

Beyond Inflammatory Bowel Disease and Irritable Bowel Syndrome, a larger classification of conditions labelled immune-mediated inflammatory disorders (IMID) behave similarly and have similar need for novel approaches to management. IMIDs include inflammatory bowel disease as well as other GI diseases like gastroesophageal reflux disease and Barrett's esophagus. IMIDs also include rheumatoid arthritis (RA), osteoarthritis, the spondyloarthritis disease spectrum, connective tissue disorders, cutaneous inflammatory conditions such as psoriasis and atopic dermatitis, asthma, ADHD, Type 1 diabetes, and autoimmune neurological diseases such as multiple sclerosis¹⁹. As a group of conditions, IMIDs have an estimated incidence of 80+ per 100,000 and affect 3-5%+ of the US population. Although IMIDs share common underlying pathogenetic features (‘public’ immune pathways), they also present unique (‘private’) pathways that define their clinical phenotype, age and sex distribution, tissue localization and therapeutic response profile, among other characteristics. The genetic overlap between IMIDs can be illustrated by the fact that of the >100 genetic loci associated with RA, only two appear specific to RA and not associated with other IMIDs. Similarly, both UC and Crohn's involve a complex interaction between genes and environment¹². Although there has been progress in identifying come genes responsible for predisposition to IBD²⁵, for example, environmental factors which may trigger the exacerbation of symptoms and subsequent relapses are not clearly understood. One of the most important factors shaping the diversity and composition of enteric flora, diet may have the potential to be a preventive and therapeutic strategy²⁰. For example, epidemiological studies suggest that polyunsaturated fatty acids may have a positive effect on rheumatoid arthritis development or the course of multiple sclerosis through their anti-inflammatory and antioxidant properties^{19,22}. As a further example, the Mediterranean Diet, Specific Carbohydrate Diet and even veganism have shown promise for the management of rheumatoid arthritis and psoriasis²¹. Given these results, it's unsurprising that IMIDs are strongly associated with a significant change in normal dietary patters and that the variation is disease-specific²³. Furthermore, psychology interventions such as cognitive behavioral therapy and self-regulation modalities have been shown to have promise as therapeutic tools, not only to provide relief from IMID symptoms but also to address IMID-caused anxiety, depression, and post-traumatic stress disorder.

IMIDs generally have no known cures and are often accompanied by various co-morbidities (including cardiovascular disease, metabolic and bone disorders, as well as psychological disorders, amongst others); thus, these conditions pose significant systemic medical challenges as well as financial burden on the healthcare system and US economy^{19,20}. For example, rheumatoid arthritis has been estimated to impact 2-4% of the US population, 20-35% of whom had to stop working within 3 years of disease onset; compared to the general population, individuals with ankylosing spondylitis have lower employment rates, experience more disability, and are absent more frequently from work. As another example, psoriasis patients generate well over $115M in lost productivity per year as a result of their conditions.²¹

It has also been demonstrated that other environmental factors such as certain types of stress, as with depression and/or other adverse life events, result in increased symptomology in patients suffering from IMIDs such as inflammatory bowel disease (IBD). The effects of psychological stress on the systemic immune and inflammatory system are complex, and depend on both the duration and intensity of the stressor. Although in the 1950s, IBD was classified as a psychosomatic disorder with many early studies finding an association between IBD and psychiatric diagnoses,²⁶ more recently a review of 138 studies found serious flaws in the methodology. Interestingly, in contrast, Mittermaier et al reported in 2004 that patients with inactive IBD had a significantly increased chance of relapse over the next 18 months if their baseline score on the Beck's depression inventory was raised.²⁷ Other more recent studies support the association of life events with a higher risk of subsequent relapse. Bitton et al found the number of stressful life events in the preceding month to be a risk factor for relapse in a one-year prospective study of 60 patients with UC²⁸, while Mardini et al found depression and, to a lesser extent, life events to be predictors of relapse in a two-year prospective study of 18 patients with Crohn's disease.²⁹ Indeed, it is known that acute psychological stress can effect gastrointestinal motility, as well as water and ion secretion.

Given the support provided by more recent studies indicating that psychological stress may indeed be a pathogenic factor in IBD, IBS, and other IMIDs, it appears obvious, then, that stress reduction therapy may have a significant therapeutic benefit, both on reducing physical symptoms of the condition as well as addressing the psychological comorbidities associated with that condition. However, most stress reduction therapy remains informal, at best, with studies of its efficacy in patients with IBD and IMIDs relatively unknown. Results of studies that have attempted to evaluate the benefit of stress reduction remains contradictory, partly because of the difficulty in standardization of the methodologies. Due to the nature of such trials, even performing them in a blinded controlled manner is difficult, including because placebo rates of up to 40% have been observed in studies evaluating therapies for IBD and related syndromes.

For example, a study reported by Schwarz and Blanchard found that combined complementary medical treatment, including cognitive behavioral therapy, muscle relaxation techniques, and patient education, was successful in reducing stress but did not result in an improvement of IBD symptoms.³⁰ Similarly, Jantschek et al also failed to find that addition of psychosocial treatments to conventional therapy led to any reduction in the number, duration, or severity of relapses in a prospective two-year study of 108 patients with Crohn's disease.³¹ However, in contrast, Milne et al reported significant improvement in relapse rate as measured by the Crohn's disease activity index, when a stress management course supplemented conventional treatment in a one year randomized trial of 80 patients.³²

Jarrett et al looked at cognitive behavioral intervention delivered mainly by telephone and compared results, as measured by a decrease in gastrointestinal symptoms and an increase in quality of life, with both in-person therapy and usual care. The results showed that both CBT intervention delivered in-person, as well as delivered primarily by telephone, was more effective in decreasing GI symptoms and increasing quality of life than usual care alone.³³

In addition to evaluating the effect of stress to the exacerbation of symptoms associated with IMIDs, there is also a growing body of research looking at the contribution of diet in disease exacerbation and the modification of dietary intake as a valid method of IMID management. Given that microbial dysbiosis is a hallmark of many IMIDs, a large majority (60-80%) of persons with Inflammatory Bowel Disease (IBD), irritable bowel syndrome (IBS) or other immune-mediated inflammatory disorders (IMIDs) report that symptoms originate from, or are exacerbated by “trigger foods,” and modify their diets to alleviate discomfort. Although such dietary interventions appear to be a reasonable complementary treatment to traditional clinical care, there is no consensus on what diet is best for IBD, IBS or IMID patients.

Zia et al reported on a comprehensive program combining cognitive behavioral therapy with relaxation (specific relaxation strategies and lifestyle behaviors), alternative thoughts (identifying thought distortions, challenging underlying beliefs, and other strategies) and dietary strategies (composition, trigger foods, meal size or timing, and eating behaviors). IBS participants who engaged in nine sessions (1 hour each) over 13 weeks were shown to experience reduced gastrointestinal symptoms and an increased quality of life as compared to those provided with usual care.³⁴

One major challenge is that these conditions present heterogeneously, and patients often respond differently to the same food, so one-size-fits-all dietary recommendations are not optimal solutions. Additionally, popular diets claiming usefulness as an adjunct to traditional care for IBD and the like, such as the low-fermentable oligosaccharide, disaccharide, monosaccharide, and polyol diet (FODMAP), have notable limitations (e.g., cost, length, complexity) and are not personalized to an individual. In a similar fashion, the use of stress reduction techniques as an aid in the reduction of disease exacerbation is an area ripe for exploration in a holistic approach to management and treatment of IMIDs including IBD and IBS. Further, recent advances in smartphone-based digital technologies and machine-learning analysis tools present an opportunity to modernize, simplify, and increasingly personalize the standard approaches to implement and evaluate interventions, such as elimination-based dietary strategies and stress reduction modalities for IBD, IBS and IMID patients. Thus, there remains a significant need to develop new approaches for effective management of IBD and other IMIDs.

### SUMMARY OF THE INVENTION

In accordance with the purpose(s) of this invention, as embodied and broadly described herein, one aspect of the present invention relates to systems and methods of use and treatment thereof to manage IMIDs including, but not limited to, IBD and IBS and, especially, to systems and methods that can be used by the individual herself or with the support of a health coach or clinician to positively affect disease and symptoms associated thereto.

In one aspect of the present invention, a method and system is provided to identify and recommend the optimal single modality or combination of modalities of intervention, including but not limited to diet, psychology, sleep and exercise to manage an individual's IMID (including but not limited to IBD and IBS), and positively impact disease symptoms as well as an individual's general health and wellbeing.

In another aspect of the present invention, a method and system is provided to identify, confirm and eliminate trigger foods included in a person's diet which are associated with exacerbation of symptoms in an individual with an IMID such as, but not limited to, IBS or IBD.

Expanding upon this aspect of the present invention, a method and system are provided to guide an individual in a personalized dietary intervention to eliminate trigger food(s), thereby delivering and maintaining potential therapeutic benefits to a participant including the amelioration, reduction, or elimination of symptoms

In yet another aspect of the present invention a system and methods are provided which include modalities generally classified as psychological therapy or as a psychology modality and which generally refers to the practice of helping an individual identify and change troubling emotions, thoughts, behaviors and the like to improve health and wellness.

A further embodiment of a psychology modality encompasses behavior modification techniques including, but not limited to cognitive behavioral therapy (CBT).

In one aspect, the psychology modality includes but is not limited to acceptance and commitment therapy, which encompasses an action-oriented approach to psychology that teaches participants to stop avoiding, denying, and struggling with their inner emotions and, instead, accept that these deeper feelings are appropriate responses to certain situations that should not prevent them from moving forward in their lives.

A further aspect of the present invention encompasses other psychology modalities including, but not limited to exposure therapy, which refers to a modality that encourages the systematic confrontation of feared stimuli, which can be external (e.g., feared objects, activities, situations) or internal (e.g., feared thoughts, physical sensations) with the goal to reduce the person's fearful reaction to the stimulus.

In yet another aspect of the present invention, modalities related to sleep hygiene are contemplated including, but not limited to behaviors and habits that influence quality, length, depth and other variables related to an individual's sleep with the goal of improving the quality of a participant's sleep and thus improving symptoms and clinical signs associated with IBS, IBD, and IMID.

A further embodiment of the present invention encompasses modalities related generally to physical exercise such as an individual's movement and activity, and encompasses behaviors, habits, quality, quantity, length and strenuousness of that activity.

In another embodiment of the present invention, methods and techniques for reducing stress are provided including, but not limited to relaxation techniques such as gut-directed hypnosis, progressive muscle relaxation; hypnotherapy; diaphragmatic breathing, mindfulness based stress reduction techniques; breathing exercises; guided meditation; guided imagery and/or visualization; yoga; Tai Chi; biofeedback; music therapy; and the like.

A further embodiment of the present invention contemplates a system which allows the input and collection of data, the analysis of that data, and a means to communicate outputs, including generation and communicating personalized treatments to allow an individual to beneficially manage IBD, IBS and/or other IMIDs, either in a self-guided manner or with the participation of an external coach, clinician or other third party whether coaching, advice or other participation is provided via written, oral, video or other media.

Another embodiment of the present invention provides a system and methods used to create a personalized care management program and protocol having stages of variable duration, which are contemplated to be iterative and are useful for a person suffering from an IMID, such as IBD and/or IBS, to positively affect symptom management and reduce exacerbation of disease symptoms. In one embodiment, the variable length program is contemplated to last approximately nine (9) weeks, but it should be appreciated that the length of any individual modality, as well as the program itself wherein single or multiple modalities are presented to an individual, may be increased or decreased in accordance with factors such as the severity and breadth of the person's symptoms, the desire of the person to engage in more than one modality, the response of the person to the various modalities as evidenced by increase in well-being, and the like.

Yet a further embodiment of the present invention contemplates a digital personalized management tool developed using an iterative methodology by which an individual with IBD, IBS and/or IMID can further refine identification and confirmation of specific food triggers to provide an individualized diet which eliminates the confirmed trigger foods while monitoring improvement in symptomology, well-being and the like, thereby improving the management of IBD, IBS and/or IMID.

In another embodiment, the digital management tool may also, separately or as a part of the dietary module, gather information, data, and the like to refine and evaluate the results of the person's participation in one or more symptom amelioration modalities, such as stress reduction, psychology, sleep, and the like, to identify the most effective symptom management techniques and practices to aid the participant in positively managing symptoms associated with the IMID condition.

Additional advantages of the invention will be set forth, in part, in the description which follows and, in part, will be obvious from the description, or may be learned by practice of the invention without undue experimentation. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate embodiment(s) of the invention and, together with the description, serve to explain the principles of the invention.

FIG. 1 is an overview of a system in accordance with a method of the present invention whereby individual health information and other inputs are gathered, provided, stored and analyzed with the results provided as an output to the participant using the method.

### DETAILED DESCRIPTION OF THE INVENTION

### Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims. Unless defined otherwise, all technical and scientific terms used herein have the same meaning as commonly understood by one of ordinary skill in the art to which this disclosure belongs. Any compositions, methods, and materials similar or equivalent to those described herein can be used in the practice or testing of the present invention. All publications cited herein are incorporated by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein.

Use of the term “about” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. It is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The use of the alternative (e.g., “or”) should be understood to mean either one, both, or any combination thereof of the alternatives.

The term “and/or” should be understood to mean either one, or both of the alternatives.

Throughout this specification, unless the context requires otherwise, the words “comprise”, “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “flare” or “flare up” is used generally to refer to a period of acute exacerbated symptoms.

A “subject,” “individual,” “person,” “participant,” “user,” or “patient” as used herein may be used interchangeably and includes any person that can use the program, the system, and the methods contemplated herein to reduce or ameliorate the symptoms associated with any IMID generally, IBD and IBS specifically, or combination thereof.

As used herein “IBD” (Inflammatory Bowel Diseases) and “IBS” (Irritable Bowel Syndrome) are used to generally refer to clinical syndromes involving inflammation of the gastrointestinal tract and include Crohn's Disease including, but not limited to, ileocolitis, illeitis, ileocolitis, jejunoileitis granulomatous colitis, and ulcerative colitis, including ulcerative proctitis, proctosigmoiditis, left-sided colitis and pancolitis, and may be accompanied by a variety of physical signs including, but not limited to, mild to intense abdominal pain, cramps, diarrhea, fistula formation, rectal bleeding, abscess formation, formation of ulcers around the anus, skin lesions and joint pain. Further, it is contemplated that the term IBS includes any IBS subtype, including IBS-C(constipation), IBS-D (diarrhea), IBS-M (mixed bowel pattern) and IBS-U (unsubgrouped).

As used herein “IMID” (immune-mediated inflammatory disorders) includes but is not limited to GI diseases like gastroesophageal reflux disease (GERD) and Barrett's esophagus, osteoarthritis, rheumatoid arthritis, the spondyloarthritis disease spectrum, connective tissue disorders, cutaneous inflammatory conditions (including psoriasis, eczema and atopic dermatitis), inflammatory bowel disease (IBD), cutaneous inflammatory conditions such as psoriasis and atopic dermatitis, connective tissue disorders, asthma, ADHD (attention deficit hyperactivity disorder), Type 1 diabetes, and autoimmune neurological diseases such as multiple sclerosis.

As used herein terms such as “amelioration,” “ameliorate,” “therapy, “treatment” or “treating,” refer to any beneficial or desirable effect associated with a reduction in one or more symptoms or other effects of a disease or condition disclosed herein, such as IBD, IBS or IMIDs in general. For example, in relation to embodiments comprising the treatment of IBD in a subject, “treatment” or “treating” includes any beneficial or desirable effect associated with a reduction in diarrhea, cramping, abdominal pain, frequency of bowel movements, and the like, but may also include an increase in subjective indicators such as a feeling of well-being and the presence of more energy. “Treatment” does not necessarily indicate complete eradication or cure of the disease or condition, or associated symptoms thereof.

As used herein, the term “modality” or “module” is interchangeable with “program” and refers to at least one particular therapeutic technique, exercise, lesson, education, method, system or process for use by a person with an IMID such as IBS or IBD to reduce or ameliorate her signs and symptoms and improve her sense of well-being. In broad terms, “modality” or “module” may also be used to refer to a specific embodiment included in the invention such as, for example, the dietary intervention modality, the behavior modification modality, and the like. Broadly, the term encompasses techniques; programs accessed by the participant; means for collecting, processing and reporting data; means for communication of that data; related software required to perform the various functions; and the like needed to have a functional application for the participant to access and use in accordance with the present invention. A specific modality may be accessed alone or in combination with at least one other modality in a program to improve the management of IBS, IBD, or an IMID. For example, in one embodiment of the present invention, a modality is provided to help the user identify, confirm, and eliminate trigger foods associated with an increase in the severity of signs and clinical symptoms associated with the condition of interest. Another modality contemplated by the present invention relates to techniques useful for stress reduction including, but not limited to CBT, hypnotherapy, guided meditation, and the like. Yet another embodiment contemplates the concurrent use of multiple modalities for any one user. Any number and/or combinations of modalities are also contemplated to be encompassed by the system and methods of the present invention.

The term “database” refers to a collection of information, data, content and the like whether gathered prior to, during, or after the program from the participant herself or on her behalf, as with data from medical records; entered by others involved with her participation, such as data from fitness watches and the like; data and content obtained from the participant's interaction with the program; as well as data obtained from independent sources such as, for example, dietary suggestions gathered from a literature review as to foods most commonly associated with IBS, IBD and IMIDs. Results of analysis of that data, information and content, communication with the participant and others, as well as content related to the participant's interactions with and progress through the program are also intended to be encompassed within the term “database.

As used herein lifestyle modification choices refer to changes or modifications that are made by the user including but not limited dietary, behavioral, sleep, exercise-related changes and the like.

As used herein, the term “psychotherapy,” “psychology” or “psychological therapy” refer to the practice, in general, of helping an individual identify and change troubling emotions, thoughts, behaviors and the like to improve health and wellness and, specifically, encompass a number of practices including but not limited to counseling, hynotheraphy, Cognitive Behavioral Therapy (CBT), stress reduction techniques and the like.

As used herein the term “Cognitive Behavioral Therapy (CBT)” refers to a form of psychological treatment that has been demonstrated to be effective for a range of problems including depression and anxiety disorders and leads to significant improvement in functioning and quality of life for many. According to the American Psychological Association, CBT is based on several core principles including that psychological problems are based, in part, on faulty or unhelpful ways of thinking and/or, at least in part, on learned patterns of unhelpful behavior. CBT treatment usually involves efforts to change thinking and behavior patterns using various strategies including learning to recognize one's distortions in thinking that are creating problems, and then to reevaluate them in light of reality.

As used herein, “stress reduction techniques” includes any technique used by itself or in combination with other techniques for reducing stress in a participant as measured by subjective criteria including, but not limited to, relaxation techniques such as progressive muscle relaxation; hypnotherapy; mindfulness based stress reduction techniques; breathing exercises; meditation; guided imagery and/or visualization; yoga; Tai Chi; biofeedback; music therapy; and the like. All of the techniques have in common the ability to reduce stress, anxiety, and depression and improve quality of life in the participant.

As used herein the term “hypnotherapy” refers to a form of therapy that uses hypnotic suggestion and guided imagery to encourage positive change, from easing stress and encouraging relaxation to promoting positive outcomes such as reduction in anxiety and relief from pain.

As used herein, “progressive muscle relaxation” is a technique that focuses on specific parts of the body and by tensing and relaxing muscles in the specific areas of the body, the participant is able to move into a more effective state of relaxation. The technique also facilitates learning the differences in sensation between tension and relaxation to aid the participant in identifying triggers leading to tension early enough to interrupt the cycle, thereby reducing deleterious effects associated with tension.

As used herein, “diaphragmatic breathing” refers to a breathing technique that focuses on the stomach, rather than the chest. Deliberately paying attention to each breath has been shown to distract and quiet the mind.

As used herein, the term “acceptance and commitment therapy” encompasses an action-oriented approach to psychology that stems from traditional behavior therapy and cognitive behavioral therapy. Utilizing acceptance and commitment therapy, participants learn to stop avoiding, denying, and struggling with their inner emotions and, instead, accept that these deeper feelings are appropriate responses to certain situations that should not prevent them from moving forward in their lives.

As used herein, the term “exposure therapy” refers to a modality of psychological therapy that encourages the systematic confrontation of feared stimuli, which can be external (e.g., feared objects, activities, situations) or internal (e.g., feared thoughts, physical sensations). The goal of exposure therapy is to reduce the person's fearful reaction to the stimulus.

As used herein, the term “sleep hygiene” refers to the behaviors and habits that influence quality, length, depth and other variables related to an individual's sleep. Addressing the behavioral and environmental context around sleep can improve quality of sleep.

As used herein, the term “physical exercise” refers to an individual's movement and activity, and encompasses the behaviors, habits, quality, quantity, length and strenuousness of that activity.

The term “symptom” as used herein, generally refers to subjective evidence of a disease or disorder, i.e., particularly a feature that is apparent to the individual with the disease or disorder such as diarrhea or vomiting and as distinguished from the term “clinical sign” which relates to objective evidence of a disease or condition, such as fever or the result of a laboratory test. However, as used herein, the terms “symptom” and “clinical sign” may be used interchangeably.

As used herein, “prevent,” “prevented,” “preventing” and the like indicate an approach for preventing, inhibiting, or reducing the likelihood of the occurrence or recurrence of one or more symptoms or other effects of a disease or condition such as IBD (including Crohn's Disease and Ulcerative Colitis), IBS or IMIDs. For example, in embodiments that relate to treating IBD in a subject, “prevent,” and similar words such as “prevented,” “preventing” and the like indicate an approach for preventing, inhibiting, or reducing the likelihood of the occurrence of clinical signs and symptoms associated with IBD. Also encompassed within these terms is the delay of the onset or recurrence of a disease or condition or delaying the occurrence or recurrence of the symptoms.

The words “preferred” and “preferably” refer to embodiments of the invention that may afford certain benefits under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

As used herein, “management” or “controlling” one or more symptoms or effects of a disease or condition (e.g., Crohn's Disease) refers to the use of the system or methods contemplated herein to improve the quality of life for an individual by providing better control of symptoms associated with the disease or condition, including diarrhea, cramping, abdominal pain, frequency of bowel movements, and the like.

As used herein, “trigger foods” refer to commonly associated foods that are known, postulated, or identified with the present invention to be associated with an increase in the severity of signs and clinical symptoms associated with IMIDs, IBD (Crohn's Disease and Ulcerative Colitis), IBS and combinations thereof, including but not limited to, rheumatoid arthritis, psoriasis, connective tissue disorders, asthma and multiple sclerosis. The term “trigger” may be used interchangeably with “trigger foods” or may be used in a broader sense to also encompass one or more specific behaviors associated with a detrimental effect in the participant. “Trigger foods” include, but are not limited to, vegetables including legumes like beans, peas, and the like; nightshades, such as tomato, peppers and the like; gluten containing foods including bread, pasta, noodles, cereal, and the like; dairy and lactose-containing foods such as milk and cheese; spicy foods; fried foods; and alcohol. One of ordinary skill in the art will understand that the foods listed above are provided for description and that the disclosure is not limited to the embodiments described but is for the purpose of describing certain embodiments only.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements. The description exemplifies illustrative embodiments. In several places throughout the application, guidance is provided through lists of examples, which examples can be used in various combinations. In each instance, the recited list serves only as a representative group and should not be interpreted as an exclusive list. All headings are for the convenience of the reader and should not be used to limit the meaning of the text that follows the heading, unless so specified. It is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in any appropriately detailed structure.

The present invention is directed to methods and systems useful for developing a more personalized and effective management of IMIDs, such as IBD and IBS, as evidenced by the amelioration of, or reduction in, symptoms associated with the disease in the user or of the symptoms associated by IMID-related anxiety, depression, and other mental health comorbidities. One embodiment of the present invention provides a personalized reiterative system and methods thereof to identify and reduce or eliminate specific triggers thereby reducing or improving symptoms the participant experiences as part of IBD, IBS or other IMIDs.

In one embodiment contemplated by the present invention, a system and associated methods are encompassed to provide dietary guidance for the identification and elimination of specific trigger foods. In accordance with that embodiment, it is contemplated that an individual will participate in at least one phase after an initial onboarding phase where, amongst other information, demographic data such as age, sex, and ethnicity; medical history including diagnosis, symptoms, and treatment of any condition; medication history; and the like is obtained for the individual. In another embodiment according to the present invention, following the introductory or onboarding phase, the individual will participate in four phases: an identification phase; an elimination phase; a reintroduction phase; and a maintenance phase. The individual may go through each phase or stage once or may, optionally, go through one or more of the phases in an iterative fashion. As may be appreciated by one in skill of the art, although four phases are contemplated by one embodiment of the present invention, it will be recognized that the invention may have either a larger or smaller number of phases and still come within scope of the invention.

In another embodiment of a system and methods in accordance with the present invention, modalities other than one offering dietary guidance for the identification and elimination of suspected trigger foods may be included. Each additional modality may be included either alone or in combination with at least one other modality including the modality providing dietary guidance. For example, it is contemplated that a system and method in accordance with the present invention to reduce or eliminate symptoms associated with an IMID such as IBD or IBS in a user could include both a modality to provide dietary guidance for the identification and elimination of suspected trigger foods, as well as at least one modality to reduce and manage stress and/or to modify behavior of the participant using any number of techniques including but not limited to CBT.

Also contemplated by the present invention is a management system which includes, but is not limited to i) a means for the collection of data, including data such as the occurrence and severity of symptoms self-reported by the individual as the individual participates in the different stages of the method, gathered passively from connected technology such as health wearables (watches, scales, and the like) or from other sources; ii) a means of storing the collected data in a data repository; iii) generation of an individualized database from the data collected and stored at each step or phase of the method of the present invention; iv) analysis of data in the individualized data base using at least one algorithm and at least one database of stored written/recorded/video content or additional health data; v) using the data analysis to develop at least one personalized disease management program utilizing one or more modalities of therapy (e.g., diet, psychology, behavior modification, stress reduction, sleep hygiene, and the like) for the individual to follow during at least one stage or phase of the method; and vi) communication of information during one or more stages of the present method including, but not limited to, statistics generated from the collected data; data output; and any change in symptoms or other parameter including objective and subjective indicators measured associated with the condition of concern following institution of at least one modality such as personalized diet or relaxation techniques by the user. One advantage of the present invention is that, as one of skill in the art can readily appreciate, the method is iterative; that is, a participant may repeat at least one step any number of times to achieve the desired result before proceeding through the remainder of the program. Further contemplated is that the user may use any of the techniques in the stress relaxation, sleep hygiene, physical activity or any other relevant modality either once or any number of times, and by monitoring data and subjective indicators the user can develop a personalized program most helpful in reducing stress, increasing well-being, and ameliorating symptoms resulting from IBS, IBD, and/or IMIDs.

A method in accordance with the present invention useful to ameliorate or reduce symptoms associated with IBD, IBS and/or IMIDs generally consists of four principal phases once onboarding of the participant is completed. As a means of illustration, one embodiment associated with the dietary modality according to the present invention contemplates including four phases as follows: Identification (Phase 1), Elimination (Phase 2), Reintroduction (Phase 3), and Maintenance (Phase 4) as illustrated in Table 1, as follows:

| | | |
|---|---|---|
| Phase | Week | Activity |

Input: Machine learning algorithm identifies 20-25 foods that may be
associated with an individual's IBD, IBS and/or IMID symptoms
to track and validate in phase 1-3.

| | | |
|---|---|---|
| 1 | 1-3 | Users eat normally and complete |
| Identification | | daily symptom & diet surveys |

Output: Machine learning algorithm down-selects a subset of foods
from the initial list of 20-25 foods that may be associated with an
individual's IBD, IBS and/or IMID symptoms and suggest they
remove these from their diet in Phase 2. In one embodiment,
the subset is 3 to 5 foods.

| | | |
|---|---|---|
| 2 | 1-2 | Users remove the suggested foods |
| Elimination | | from their diet entirely and |
| | | complete daily symptom & |
| | | diet surveys |

Output: Participants establish a baseline to validate that foods
identified in Phase 1 are associated with increased symptom severity
as part of Phase 3.

| | | |
|---|---|---|
| 3 | 1-2 | Every 3 days, participants |
| Reintroduction | | reintroduce another potential |
| | | trigger food and complete daily |
| | | surveys throughout, guided by a |
| | | machine learning algorithm that |
| | | evaluates daily symptoms |

Output: Final description of the least restrictive diet that may
measurably improve a participant's IBD, IBS and/or IMID symptoms

| | | |
|---|---|---|
| 4 | 2 | Users eat modified diet and |
| Maintenance | | complete daily symptom & |
| | | diet surveys |

Output: Follow-up monitoring of efficacy of recommended diet

Onboarding (pre-Phase 1): In this phase, health data is collected from each participant including but not limited to IBD, IBS and/or IMID symptomatology; medication taken; general medical history and the like. Relevant health can be gathered in a user-reported manner, from electronic medical records, medical claims databases, wearables, or other sources of external data, including but not limited to clinicians/practitioners. In addition, during the Onboarding stage the present invention contemplates the collection of other non-medical data, if desired, including but not limited to demographics such as, but not limited to gender, age, ethnicity; physical activity and the like. Also contemplated is that each participant inputs dietary preferences, communication preferences, prior experience with therapeutic modalities, and other clinical and non-clinical questions. Table 2 illustrates additional data that can be collected as follows:

Data Gathered

Third-party (privately-held, publicly-available and/or
purchased/licensed) data on: Medical advancements and clinical
databases on IBD/IBS/IMID; IBS/IBS/IMID biomarkers; dietary
intervention data; stress reduction data; individualized or aggregated
medical records; individualized or aggregated medical and
pharmaceutical claims data, and the like
Demographic and health information including age; sex; pregnancy
status; race/ethnicity; comorbidities; smoking history; surgical
history; activity of IBD, IBS & IMID symptoms; modification of
diet; experience with therapeutic modalities; length of time with
condition; participation in and parameters of participation in stress
reduction modality and the like
Program-related data gathered daily, weekly or after specific phases:
medication adherence; 24 h recall of food items eaten; disease-related
symptom activity; mental health symptom activity; self-reported
outcome (stress, energy, alertness, and the like); participation in at
least one stress reduction technique, and the like
Technology-related data: user engagement data; machine learning
optimization data; connected “wearable” technology data such as
heart rate monitors, fitness watches, connected scales, breath
testing, and the like;

Analysis includes generating and using machine learning algorithms and providing individual level data analysis; population level data analysis; and additional analysis including clustering by similar characteristics such as clinical symptoms and/or signs, behavioral manifestations, socioeconomic levels and the like. A computing device may execute one or more artificial intelligence and/or machine learning software programs to generate and update the algorithms, generate and update health scores of users, identify trends among user characteristics, or other similar analysis. One having skill in the art will recognize that the artificial intelligence and machine learning software may execute various artificial intelligence and machine learning algorithms and processes, such as generalized linear models, random forests, support vector machines, unsupervised and/or supervised clustering, and deep learning (e.g., neural networks), among others. In some examples, machine learning software may “learn” (e.g., update data processing algorithms according to historical data trends) from training data, which, in some instances, includes labeled data.

Statistical analysis is completed according to desired outputs including baseline IBD, IBS and/or IMID symptom scores for each participant as well as depression, anxiety and other mental health symptom scoring, with additional outputs generated that are useful in a method in accordance with the present invention. For example, one embodiment of the invention contemplates calculating and predicting user engagement scores or utilizing supervised and/or unsupervised learning techniques to train a user symptom score model. In this embodiment of the invention, a symptom score model applied by an operations server or other computing device of the digital service may be trained and re-trained using any number of known statistical and regression algorithms against a predetermined set of data fields. In some cases of this embodiment, a random forest regression or other similar machine learning or artificial intelligence model can be trained to predict health outcomes utilizing database records for individuals who have IBS, IBD, and/or IMID health conditions. In this exemplary symptom score model, a target variable-IBS, IBD, and/or IMID symptom scores—is continuous. In another example, a gradient boosted classifier can be trained on historical database records for individuals, to determine a likelihood of symptom amelioration. The operations server may retrain the symptom score models either with or without input from personnel of the digital service. In an automated implementation of this embodiment, the operations server may retrieve values of certain data fields from user records that are stored in a database, and may then reapply the particular training algorithm. In some cases, the values of the data fields may include symptom scores calculated for the user and/or the number of interactions between such user and the software. An administrator or other person may review the data records and manually retrain the symptom score model via a graphical user interface (GUI) by, for example, manually adjusting algorithms used to train predictive models.

Other outputs of onboarding data collection and analysis include, but are not limited to, the recommendation of personalized disease management programs including at least one therapeutic modality. In another embodiment of the invention, outputs of onboarding data collection include initial trigger food recommendations and the generation of at least one hypothesis, to be tracked, validated, and possibly modified in Phase 1 and subsequent phases, as to which foods may be associated with the production and/or worsening of symptoms associated with the individual's condition of concern. In another embodiment of the invention, outputs of statistical models may include those specific for additional modalities included in the methods according to the present invention. For example, participation in a stress reduction modality includes choosing at least one technique and generating data from the participation including but not limited to subjective scoring of parameters related to anxiety, depression, and general well-being. Standard references may be consulted to determine the type of analysis methodology to use including, for example, Introduction to Statistical Analysis (1969), Dixon and Massey; McGraw-Hill Publishing, New York and Handbook of Data Analysis (2004), Hardy and Bryman, SAGE Publications (the contents of which are incorporated by reference it their entirety).

Phase 1 (Identification): for exemplification purposes only, phases related to a dietary modality for the identification, confirmation, and elimination of trigger foods associated with the worsening of a user's symptoms associated with IBD or IBS in accordance with the present invention will be illustrated as follows: following introductory Onboarding, a method according to the present invention contemplates that a participant will enter Phase 1 (Identification) which, in one embodiment lasts approximately 3 weeks, during which time each participant eats his or her regular diet. A daily survey will be completed including questions about self-reported GI and/or IMID symptoms (using the symptom scoring methodology as appropriate based on diagnosis) and a 24-hour recall of their unique list of trigger foods eaten. In one preferred embodiment of the present invention, the survey is automated digitally for ease of compliance. A method for a program in accordance with the present invention will also incorporate communication with all participants. In one embodiment of the present invention, communication with participants may be through at least one of a) an encrypted mobile application, b) an encrypted automated texting service, and/or c) secured email or a similar encrypted tool. One embodiment of the Identification Phase (Phase 1) contemplates data inputs including participant health data; non-health participant data; and third party data such as medical test results. Daily food intake data, including tracking results associated with a diet including initial trigger foods and particularly those triggers identified in the initial hypothesis include other examples of data inputs. In one embodiment of the present invention, a participant in preparation for Phase 1 (Identification) will be assigned a unique high-potential set of 20-25 trigger foods to track, based upon personalized factors including diagnosis type, demographic data, symptom severity at intake, and the like; however, it will be recognized by one of ordinary skill that the number of proposed trigger foods may be less or more than the 20-25 foods suggested above, depending upon specific circumstances, and the length of Phase 1 may be greater or lesser than three weeks. Analysis of information collected during Phase 1 or subsequent Phases can be undertaken at various time points. Statistical analysis may include single variable, multivariable, linear or nonlinear regression; clustering by similar participant characteristics; and other methods well known to those skilled in conducting such analyses. For example, one embodiment of the present invention contemplates using a multivariate machine learning algorithm to analyze each participant's individual diet and symptomology to narrow the initial list of potential trigger foods and identify those most strongly associated with adverse symptoms reported during Phase 1. Outputs associated with Phase 1 include, but are not limited to, the refinement of the initial hypothesis as to which foods are implicated as triggers and narrowing the initial list of trigger foods. In one embodiment, the list of triggers is narrowed to 3-5 foods, hypothesized to be most likely responsible for the majority of the symptoms experienced.

Phase 2 (Elimination): In phase 2 which, in one embodiment, lasts approximately two weeks, participants are asked to eliminate the 3-5 trigger foods identified during Phase 1 from their habitual diets. In one embodiment of the Elimination Phase in accordance with the present invention, data inputs during this period of time may include participant health data; non-health participant data; and third-party data such as medical test results, similar to data collected during Phase 1. Statistical analysis may be accomplished using a variety of generally accepted methods including multivariable regression; comparative statistics such as a t-test or descriptive statistics. The present invention also contemplates that, in Phase 2, the software used as well as statistical analysis of the data and the participant herself will determine an initial validation of symptom amelioration or improvement following the elimination of certain trigger foods. It should be recognized, however, that a participant could undergo more than one round of the Elimination Phase (Phase 2), if necessary or desirable, to identify which trigger foods, when eliminated, lead to improvement.

Phase 3 (Reintroduction): Following the elimination of certain trigger foods in Phase 2 (Elimination Phase), participants are prompted to reintroduce eliminated foods in the Reintroduction Phase (Phase 3). In one embodiment of the present invention, putative trigger foods are reintroduced one at a time, every three days and wherein each day the participant increases their food intake. It is contemplated that, in one embodiment, this Phase would last about one to two weeks, but it should be recognized that depending on a number of factors including, but not limited to the participant herself, the number of trigger foods eliminated, and the symptoms reported, two weeks is an average time for this Phase to be completed. Just as in Phase 1 and 2, a participant may undergo more than one round of the Reintroduction Phase (Phase 3). Participants complete the same daily digital survey as in Phases 1 and 2. One embodiment of the Reintroduction Phase contemplates collecting data inputs such as participant health data; non-health participant data; and third party data such as medical test results, amongst other criteria. Statistical analysis is completed using methods similar to those used in earlier Phases. The present invention also contemplates that, as in the earlier Phases, software may be used for statistical analysis of the data collected. Output from this Phase includes a secondary validation of symptom impact; i.e., confirmation of identity of a trigger food if the individual reports or experiences worsening symptoms after reintroduction of that food.

Phase 4 (Maintenance): After the reintroduction phase is complete, one embodiment of the invention contemplates that participants implement the modified diet resulting from removing the trigger foods identified in Phases 1-3 for an additional two weeks. Following Phase 3 (Reintroduction), the present invention contemplates that the participants enter into the final Maintenance stage using implementation and monitoring of the final diet designed by eliminating suspected trigger foods and validating symptom amelioration. Phase 4 includes exemplary inputs including participant health data; non-health participant data; and third party data such as medical test results, amongst other criteria, as well as all data collected during Phases 1-3. Statistical methodology also includes descriptive statistics and the like with the principal output from the Maintenance Phase including the generation of a final diet to be implemented in the individual's daily life for the control, amelioration, reduction or elimination of the symptoms and clinical signs associated with the condition of concern. It should be recognized, however, that a participant can and usually will require iterative rounds of Phase 2 to Phase 4 to identify and validate the final diet recommendation.

In one embodiment of the present invention, at study midpoint (end of Phase 2) and study completion, participants also complete qualitative assessments of quality of life and other outcomes. Additional participant-reported outcomes measuring the program's impact on a user's understanding of his/her disease and ability to make digestive choices, as well as subjective evaluations of the desirability of the tool, are also assessed and integrated into algorithmic analysis to provide continually improving recommendations aimed at improving the health, wellbeing and quality of life of IBD, IBS and other individual with IMID.

It will be recognized by one of skill in the art that the present invention contemplates other embodiments including incorporation of additional phases and/or additional therapeutic modalities that may be included to aid a participant in reducing or ameliorating symptoms associated with an IMID such as IBS or IBD. For example, in one embodiment of a dietary modality as encompassed by the present invention, a Phase 0 is included which provides a list of 20-25 potential trigger foods identified by conducting a systematic literature review of efficacy studies on diets previously described, clinical practice guidelines, meta-analyses on dietary approaches to IBS and IBD management, and survey studies on the daily dietary practices of individuals with IBS and IBD. In one embodiment of the invention, for example, data from a total of 4,565 individuals with IBS, 1,560 individuals with IBD, and 2,993 health controls are included in the analysis, but the size and composition of the data used in other embodiments may differ based on the user, type of therapeutic modality, goal of therapy, or other context. Using these pooled records, an aggregated list of 246 (or another amount required by the analysis engine) frequently eliminated trigger foods (e.g., alliums, legumes, cultured dairy, and caffeine) may be generated and mapped to the aggregated clinical and demographic characteristics (e.g., diagnosis, disease subtype, age, and length of disease course) reported in each study. Guiding principles derived by this relational database can be used to supervise a set of sequential weighing, sorting and down-sampling algorithms (leveraging feedback-based recursion) to assign a user a unique set of approximately 21 high-potential trigger foods in pre-Phase 1, which is tracked by each participant in Phase 1.

Yet another embodiment contemplated by the present invention includes a system and associated methods, either alone or in combination with a dietary module, to provide psychological therapy and psychological support for individuals with IMID in order to improve IMID symptoms, concomitant psychology symptoms and improve overall well-being. For example, an embodiment of this invention may be composed of a stress-reducing modality providing the user access to a personalized recommended set of psychological techniques including, but not limited to, breathing exercises; mindfulness and mediation practice; progressive muscle relaxation; guided imagery and/or visualization; yoga and Tai Chi; biofeedback exercises; music therapy; gut directed hypnosis; diaphragmatic breathing; acceptance and commitment therapy; education about the mind and gut connection, and the like for the purpose of reducing stress and anxiety and thereby increasing the feeling of well-being and decreasing the onset and/or frequency of symptoms associated with IBD, IBS and the like. For example, in one embodiment of a psychology (stress reduction) modality as encompassed by the present invention, an initial Phase 0 is included which provides a personalized recommended therapeutic program consisting of between 20-40 modules of psychology content, grouped by type of therapy (for example, gut directed hypnosis and/or cognitive behavioral therapy), but one skilled in the art will recognize that any number of modules may be recommended, given clinical, demographic, algorithmic, engagement-related or other requirements. Recommended psychology modules for each user are identified in one aspect by reviewing a proprietary systematic database including, for example, a literature review of efficacy studies on psychotherapy, clinical practice guidelines, meta-analyses on psychology approaches to IBS and IBD management, and survey studies on the behavioral practices of individuals with IBS and IBD. Other aspects of the database include pooled records and input from board-certified gastro-psychologists. An aggregated list of over 150 (or other specified amount required by the analysis engine) high-efficacy therapeutic exercises and content modules is generated and mapped to the aggregated clinical and demographic characteristics (for example, diagnosis, disease subtype, age, and length of disease course) reported in each study. Guiding principles derived by this relational database are used to supervise a set of sequential weighing, sorting, and down-sampling algorithms to assign a user a unique set of modules. Users of this embodiment complete modules and, by collecting and analyzing the data collected from the participant, a machine learning algorithm adapts the recommended set of modules on a daily, weekly, or monthly basis based on factors such as the participant's symptomatic response or lack thereof after participation with the module(s). The system and method of the present invention thus continually adapts and improves a recommended module to provide an individualized recommendation and program to optimize the user's outcome following participation in each recommended module. As a means of illustration, one embodiment associated with the psychology modality according to the present invention contemplates the utilization of three psychology techniques in a program of 14 weeks, but it will be apparent to those skilled in the art that the process may be lengthened/shortened, utilize different types of psychology or stress reduction modules or a different number of techniques for each participant. An embodiment of the program for illustration follows:

| | | |
|---|---|---|
| Modality | Week | Activity |

Input: Machine learning algorithm identifies three types of psychological
therapy that have the highest likelihood of improving an individual's
IBD, IBS and/or IMID symptoms, as well as mental health comorbidities.

| | | |
|---|---|---|
| 1 Cognitive | 1-4 | Users complete daily symptom |
| Behavioral | | surveys, complete psychology |
| Therapy | | exercises, read educational material, |
| | | and listen to recorded psychological |
| | | therapy sessions. |

End of phase: Machine learning algorithm utilizes the symptom surveys
to suggest the most effective next psychology modality to present to the
participant (including an extension of modality #1, above)

| | | |
|---|---|---|
| 2 Acceptance and | 5-9 | Users complete daily symptom |
| Commitment | | surveys, complete psychology |
| Therapy | | exercises, read educational material, |
| | | and listen to recorded psychological |
| | | therapy sessions. |

End of phase: Machine learning algorithm utilizes the symptom surveys
to suggest the most effective next psychology modality to present to the
participant (including an extension of modality #2, above)

| | | |
|---|---|---|
| 3 Breathing and | 9-14 | Users complete daily symptom |
| Mindfulness | | surveys, complete psychology |
| | | exercises, read educational material, |
| | | and listen to recorded psychological |
| | | therapy sessions. |

End of phase: Machine learning algorithm utilizes the symptom surveys
to suggest the most effective next psychology modality to present to the
participant (including an extension of modality #3, above)

A further embodiment encompassed by the methods and system of the present invention contemplates improving a participant's IMID symptoms and overall well-being by providing psychological therapy and psychological support concomitantly with dietary support and other modalities of treatment and support, such as physical exercise, sleep hygiene exercises, direct follow up with practitioners, participation with counselors, and the like. For example, one additional modality that may be included in the methods and system according to the present invention may comprise a combination of stress-reducing modalities, diet-related modalities and/or any other modality that provides users with relief from IMID symptoms or comorbid/concomitant mental health, or other physical symptoms. In such an embodiment, the invention provides the participant with access to a personalized recommended set of psychological techniques including but not limited to breathing exercises; mindfulness and mediation practice; progressive muscle relaxation; guided imagery and/or visualization; yoga and Tai Chi; biofeedback exercises; music therapy; gut directed hypnosis; diaphragmatic breathing; acceptance and commitment therapy; education about the mind and gut connection, and the like; dietary guidance for the identification and elimination of specific trigger foods; techniques for the improvement of sleep hygiene and quality; exercises and support to improve physical activity; or any number of other modalities that, when a participant uses the modality, may increase the feeling her well-being and/or decreases the onset and/or frequency of symptoms associated with IBD, IBS and the like. For example, in one embodiment of a multi-modality disease management program as encompassed by the present invention, an initial pre-Phase 1 is included providing a personalized recommended therapeutic program consisting of between 1-100+ modules such as psychological therapy, relaxation therapy, behavior modification techniques, dietary management, sleep therapy, physical activity content and the like, which permits a participant to access the modules individually, in combination, or even grouped by type of therapy. One skilled in the art will recognize that any number of modules may be recommended based on factors including but not limited to clinical status, demographic data, algorithmic data, engagement-related parameters and other requirements. In one embodiment of the present invention, recommended disease management modules for each user are identified by factors including but not limited to conducting a systematic literature review of efficacy studies on each therapeutic modality to identify relevant modalities, clinical practice guidelines, meta-analyses on each modality's impact on IBS, IBD and IMID management, survey studies on the behavioral practices of individuals with IBS, IBD or IMID and the like. For example, pooled records aid in providing an aggregated list of several hundred high-efficacy therapeutic exercises and content modules generated from the database and mapped to the aggregated clinical and demographic characteristics associated with a particular disease or syndrome (e.g., diagnosis, disease subtype, age, and length of disease course) reported in each study. Guiding principles derived by this relational database are used to supervise a set of sequential weighing, sorting, and down-sampling algorithms to assign a user a unique set of modules. Users of this embodiment complete at least one module but more likely a combination of modules during which a machine learning algorithm adapts the recommended set of modules to complete on a daily, weekly or monthly basis based on the participant's individualized factors including symptomatic response or lack thereof. As a means of illustration, one embodiment associated with the psychology and dietary modalities according to the present invention contemplates the utilization of three psychology techniques and four phases of dietary therapy in a program of approximately 18 weeks, but it will be apparent to those skilled in the art that the program may be lengthened/shortened, utilize different modalities of therapy, or include a different number of techniques for each participant, and that the length of each phase may not be the same for each modality. The following chart is a summary of one such embodiment provided for illustration as follows:

| | | |
|---|---|---|
| Phase & | | |
| Modality | Week | Activity |

Input: Machine learning algorithm identifies three types of psychological
therapy that have the highest likelihood of improving an individual's
IBD, IBS and/or IMID symptoms, as well as mental health comorbidities.
A second machine learning algorithm identifies 20-25 foods that may be
associated with an individual's IBD, IBS and/or IMID symptoms to
track and validate in phase 1-3.

| | | |
|---|---|---|
| 1 Diet | 1-4 | Users eat normally and complete daily |
| Identification | | symptom & diet surveys |
| 1 Cognitive | | Users complete daily symptom surveys, |
| Behavioral | | complete psychology exercises, read |
| Therapy | | educational material, and listen to |
| | | recorded psychological therapy sessions. |

Output: Machine learning algorithm down-selects a subset of foods from
the initial list of 20-25 foods that may be associated with an individual's
IBD, IBS and/or IMID symptoms and suggest they remove these from
their diet in Phase 2. In one embodiment, the subset is 3 to 5 foods.
End of phase: Machine learning algorithm utilizes the symptom surveys
to suggest the most effective next psychology modality to present to the
participant (including an extension of modality #1, above)

| | | |
|---|---|---|
| 2 Diet | 5-9 | Users remove the suggested foods from their |
| Elimination | | diet entirely and complete daily |
| | | symptom & diet surveys |
| 2 Acceptance and | | Users complete daily symptom surveys, |
| Commitment | | complete psychology exercises, read |
| Therapy | | educational material, and listen to |
| | | recorded psychological therapy sessions. |

Output: Participants establish a baseline to validate that foods identified
in Phase 1 are associated with increased symptom severity as part of
Phase 3.
End of phase: Machine learning algorithm utilizes the symptom surveys
to suggest the most effective next psychology modality to present to the
participant (including an extension of modality #2, above)

| | | |
|---|---|---|
| 3 Diet | 9-14 | Every 3 days, participants reintroduce another |
| Reintroduction | | potential trigger food and complete daily |
| | | surveys throughout, guided by a machine |
| | | learning algorithm that evaluates |
| | | daily symptoms |
| 3 Breathing and | | Users complete daily symptom surveys, |
| Mindfulness | | complete psychology exercises, read |
| | | educational material, and listen to |
| | | recorded psychological therapy sessions. |

Output: Final description of the least restrictive diet that may measurably
improve a participant's IBD, IBS and/or IMID symptoms
End of phase: Machine learning algorithm utilizes the symptom surveys
to suggest the most effective next psychology modality to present to the
participant (including an extension of modality #3, above)

| | | |
|---|---|---|
| 4 Diet | 15-18 | Users eat modified diet and complete daily |
| Reintroduction | | symptom & diet surveys |
| 4 | | Users complete daily symptom surveys, |
| Gut-Directed | | complete psychology exercises, read |
| Hypnosis | | educational material, and listen to |
| | | recorded psychological therapy sessions. |

Output: Follow-up monitoring of efficacy of recommended
diet & psychology

Other modalities contemplated to be encompassed by the present invention include, but are not limited to, incorporation of a module for an individualized program and database to monitor and track useful parameters including physical activity/fitness of a participant including by measuring and tracking factors including steps, heart rate, percentage of time spent standing up versus sitting down, recovery rate for physical activity, VO2 max, and the like. The invention also contemplates tracking and providing feedback for other features associated with general well-being including, but not limited to sleep patterns by monitoring and reporting to the participant and, optionally, to a caregiver or coach, length of sleep, percentage of time in each phase of sleep, quality of sleep, and the like.

In yet a further embodiment of the present invention, the contemplated method may be reiterative. That is, a participant may pass through at least one Phase more than once or participate in more than one modality or technique in a modality to achieve desired results.

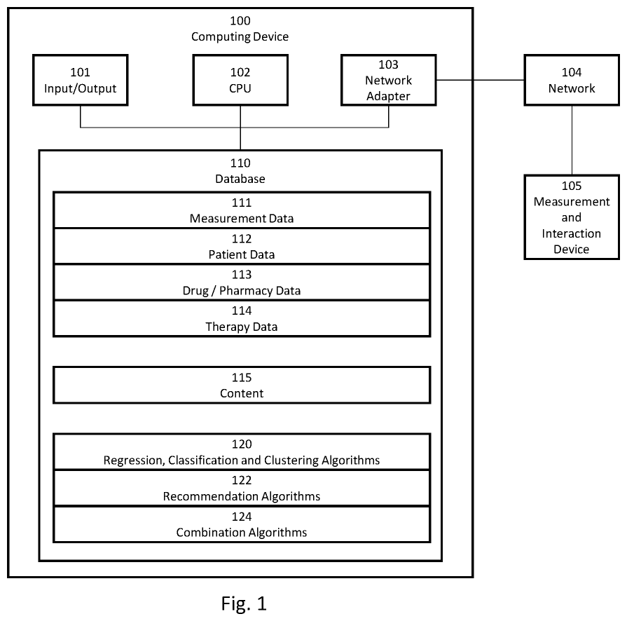

Referring now to FIG. 1, it is pointed out that like reference characters designate like or similar parts throughout the drawings. The Figures, or drawings, are not intended to be to scale. For example, purely for the sake of greater clarity in the drawings, component size and spacing are not dimensioned as they actually exist in the assembled embodiment.

Computer Readable Programming: Many operating systems, including Linux, UNIX®, OS/2®, and Windows®, are capable of running many tasks at the same time and are called multitasking operating systems. Multi-tasking is the ability of an operating system to execute more than one executable at the same time. Each executable is running in its own address space, meaning that the executables have no way to share any of their memory. Thus, it is impossible for any program to damage the execution of any of the other programs running on the system. However, the programs have no way to exchange any information except through the operating system (or by reading files stored on the file system).

Multi-process computing is similar to multi-tasking computing, as the terms task and process are often used interchangeably, although some operating systems make a distinction between the two. The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device.

The computer readable storage medium may be, for example, but is not limited to an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing.

An example of a system is illustrated in FIG. 1. A computing device 100 is depicted along with a processing unit 102 (e.g. a central processing unit (CPU), but also encompassing graphics processing units (GPUs) or even multiple processors or cores), an input/output device 101, a network adapter 103, and database 110. The network adapter 103 connects the computing device 100 to a network 104 which may include a measurement and interaction device 105. Within the database 110 of the computing device 100 reside data including, but not limited to personalized user data 112 including results of physical exams including blood panels, histology/pathology results; endoscopy data including images, videos, annotations on those videos, stool/microbiome testing, sweat, testing of breath/air such as spirometer testing, gut permeability, as well as results of imaging including X-Rays, MRI, CAT scan, fluorescence and the like. Other data contemplated to reside within the database 110 of the computing device 100 include drug/pharmacy data 113 comprising information about prescription drugs; over-the-counter medicines and nutritional supplements that may be taken by the participant. It is also contemplated that database 110 of the computing device 100 will comprise measurement data 111 including, but not limited to information from surveys; behavioral health data; data from wearable devices, such as watches, as well as data gathered by heart rate monitors, scales, connected toilets, sensors, environmental sensors and the like are contemplated to be encompassed within the present invention and may be collected. In another embodiment, data, such as obtained through electronic medical record/electronic health record (EHR/EMR), individual health records may also be collected and used to design and modify an individualized program for the participant to positively impact management of IBS, IBD and IMID. In addition to measurement data 111, user data 112, drug/pharmacy data 113, additional data may be gathered and stored such as therapy data 114 as collected from the participant's interaction with and progress through the psychology, behavior modification, dietary modification and other therapeutic modalities and programs. Content, including but not limited to, a database of therapeutic treatments, therapeutic exercises, recorded audio/video, or written material is gathered and stored in 115. Some data may reside in other locations connected to the network including, but not limited to a database of clinical practice guidelines; a database associated with behavioral data; a database of published research and the like. Also in the database 110 of the computing device may reside various programs, sub-routines or algorithms such as classification algorithms 120, recommendation algorithms 122, analysis and comparison algorithms 124, machine learning algorithms (ML), artificial intelligence (AI) algorithms, and combinations thereof. ML algorithms are a part of artificial intelligence and use an assortment of accurate, probabilistic, and upgraded techniques that empower computers to pick up from the past point of reference and perceive hard-to-perceive patterns from massive, noisy, or complex datasets. AI algorithms are a set of instructions or rules that enable machines to learn, analyze data and make decisions based on that knowledge. These algorithms can perform tasks that would typically require human intelligence, such as recognizing patterns, understanding natural language, problem-solving and decision-making.

A computer readable storage medium, as used herein, is not to be construed as being transitory signals per se, such as radio waves or other freely propagating electromagnetic waves, electromagnetic waves propagating through a waveguide or other transmission media (for example, light pulses passing through a fiber-optic cable), or electrical signals transmitted through a wire. Computer readable program instructions described herein can be downloaded to respective computing/processing devices from a computer readable storage medium or to an external computer or external storage device via a network 104, for example, the Internet, a local area network, a wide area network and/or a wireless network. The network 104 may comprise copper transmission cables, optical transmission fibers, wireless transmission, routers, firewalls, switches, gateway computers, and/or edge servers. A network adapter card or network interface 103 in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions. These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. The flowchart and block diagrams as in FIG. 1 illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s).

In alternative embodiments, the functions noted in the blocks may occur out of the order noted in the Figure. For example, two blocks shown in succession may, in fact, be executed substantially concurrently, or in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or that carry out combinations of special purpose hardware and computer instructions. Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

From the above description, it can be seen that the present invention provides a system, computer program product, and method for the efficient execution of the described methods and techniques. References in the claims to an element in the singular is not intended to mean “one and only” unless explicitly so stated, but rather “one or more.” All structural and functional equivalents to the elements of the above-described exemplary embodiment that are currently known or later come to be known to those of ordinary skill in the art are intended to be encompassed by the present claims. No claim element herein is to be construed under the provisions of 35 U.S.C. section 112, sixth paragraph, unless the element is expressly recited using the phrase “means for” or “step for.”

The present invention now will be described more fully by the following examples. This invention may, however, be embodied in many different forms and should not be construed as limited to the embodiments set forth herein; rather, these embodiments are provided so that this disclosure will be thorough and complete, and will fully convey the scope of the invention to those skilled in the art. It is to be understood that the particular examples, materials, amounts, and procedures are to be interpreted broadly in accordance with the scope and spirit of the invention as set forth herein.

### EXAMPLES

### Example 1: Personalized Elimination Diet for Self-Management of Inflammatory Bowel Disease (IBD) and Irritable Bowel Syndrome (IBS)

A decentralized single-center open-labeled uncontrolled prospective cohort study was conducted over nine weeks (63 days). All participants provided written informed consent and received financial compensation and the study and all associated materials were approved by the Advarra Institutional Review Board (protocol #50728). Adults 18-65 years of age previously diagnosed by a gastroenterologist with IBS (meeting Rome III criteria for any subtype of IBS), Crohn's Disease, or Ulcerative Colitis (established by the endoscopic, histologic, and radiologic criteria) or both, with active symptoms, were eligible for inclusion. Minimum symptom activity was determined by the appropriate condition-specific symptom severity score: IBS-SSS≥150 for IBS, sCDAI≥175 and <400 for Crohn's Disease, and p-SCCAI≥2 and <12 for Ulcerative Colitis. Exclusion criteria included pregnancy, significant comorbidities, smoking and disordered eating history, ostomy or known symptomatic intestinal stricture, current use of altered diet, and recent start or change in dose of GI medication.

### Example 2: Intervention

Over a period of nine weeks, participants were guided through four experimental phases of the personalized elimination diet: identification, elimination, reintroduction and maintenance. A link to a secure digital survey was sent to participants each morning of the study period via encrypted text message/SMS and the data was analyzed using machine learning to dynamically guide users from phase to phase in the following protocol.

Based on a meta-analysis of the literature on the dietary practices of individuals with IBD and IBS and the diets described previously, the study team generated a database of 246 frequently eliminated “trigger” foods. Based on diagnosis type, demographic data, symptom severity at intake and prior research cited above, each participant was assigned a unique high-potential set of 21 trigger foods to track in phase 1 using a machine-learning algorithm in the onboarding phase.

During phase 1 (3 weeks), participants ate their regular diet and completed a daily digital survey with questions about self-reported GI symptoms (IBS-SSS, p-SCCAI, and mHI-CD as appropriate based on diagnosis) and a 24-hour recall of their unique list of 21 trigger foods eaten. Because there is significant (30-40%) overlap between IBD and IBS diagnosis reported in the literature, participants with IBD completed both the IBS-SSS and the appropriate IBD score.

Between phase 1 and phase 2, a multivariate machine learning algorithm was used to analyze each participant's individual diet and symptom data to identify the 3-5 trigger foods most strongly associated with adverse symptoms from the initial list of 21. In phase 2 (2 weeks), participants were asked to eliminate these 3-5 trigger foods from their habitual diets and complete the same daily digital survey, abbreviated to only include this smaller subset of foods. Following phase 2 (elimination), participants were prompted to reintroduce eliminated foods in phase 3, one at a time, every three days, each day increasing food intake by one serving (˜1-2 weeks depending on the individual). Participants completed the same daily digital survey as in phase 1 and 2. If daily symptoms increased in severity participants were prompted to pause the reintroduction for 3 days or until symptoms abated to phase 2 baseline values. After reintroduction, participants implemented the modified diet that they had created for themselves (i.e. removing the trigger foods identified in phase 1-3) for an additional 2 weeks in phase 4.

At study midpoint (end of phase 2) and at completion, participants completed qualitative assessments of their energy/alertness, stress, physical activity, and quality of life. Additional user-reported outcomes measuring the program's impact on an individual's understanding of his/her disease and ability to make digestive choices, as well as subjective evaluations of the desirability of the tool were assessed.

### Example 3: Outcome Measures, Statistics and Results

Primary and secondary endpoints were collected at midpoint (week 5) and study completion (week 9). The primary outcome was symptomatic improvement measured in four ways: statistical and clinical significance of symptom improvement, achievement of remission and persistence of symptom amelioration. Symptoms were measured by the appropriate symptom severity score (P-SCCAI for UC, mHI-CD for CD and IBS-SSS for IBS). Statistically significant changes were evaluated by the appropriate statistical analysis. Maintenance of symptom improvement from baseline to end of study were measured to evaluate persistence. Additional self-reported outcomes were also measured, including impact on disease knowledge, overall wellbeing, and quality of life. For these metrics, a majority (>60%) of participants responding “Agree” or “Strongly Agree” in a 5-point Likert scale was considered successful.

Secondary endpoints related to this program's feasibility and desirability. Feasibility was measured by participant engagement, retention, and adherence to the program. Engagement was measured by daily completion of surveys and participant retention. Adherence to study protocol was measured by the percentage reduction of trigger food intake and number of days compliant with program recommendations.

Desirability was measured via qualitative satisfaction scores (0-100%) and Net Promoter Score (NPS). The NPS categorizes responders into 3 groups: “promoters” who recommend the tool, “passives” who are happy but wouldn't actively promote it, and “detractors” who actively discourage others to use it. NPS scores were calculated by subtracting the percentage of detractors from the percentage of promoters (score range −100 to +100). A NPS score ≥0 and a user satisfaction score >50% was defined as “desirable”.

Statistical Analysis: To evaluate primary endpoints, repeated measures ANOVAs with post hoc, Bonferroni-corrected, two-tailed paired-samples t-tests were used to compare symptom severity in phase 1 to symptom severity following trigger food elimination and maintenance (phase 2 and 4). Statistical analysis was conducted at both the individual-level and group-level. Descriptive statistics were reported as averages, medians, counts or percentages. Qualitative data were analyzed using McNemar's Chi-square test with Yates' continuity correction. A p value of <0.05 was considered statistically significant for primary and secondary outcomes

To evaluate secondary endpoints, the appropriate descriptive statistics were used; additional qualitative data were analyzed using McNemar's Chi-square test with Yates' continuity correction. All statistical analyses were conducted using web based software known as STATA and JASP.

In one embodiment, statistical analysis was performed prior to program start (pre-phase 1), between phase 1 and phase 2, at the end of phase 2, and daily during phase 3. Prior to study start, a machine learning algorithm determines a set of high-potential trigger foods (“hypothesis”), given the user's unique biological, clinical and socioeconomic characteristics, data gathered from other users and third-party sources. Between phase 1 and 2, a second machine learning algorithm for each participant is used to identify the subset of food categories hypothesized prior to phase 1 to be most likely to cause GI symptoms. At the end of phase 2, a t-test (or other relevant statistical method) can be used to compare symptom severity during phase 1 to symptom severity after trigger food elimination, to verify if symptoms have improved. Finally, a t-test (or other relevant statistical method) can be used to compare symptom severity during the 3 days of trigger food reintroduction in phase 3 to the average symptom severity at the end of phase 2 (i.e. baseline), to verify if symptoms have returned. Additional statistical analysis can be conducted as necessary and desired. At study completion, standard statistical descriptors (such as mean, standard deviation, median and others) can be determined to measure participant engagement. Principal factor analysis can be conducted to identify groupings in participant characteristics/demographics to understand which participants responded best to the protocol used.

Related to data collection, management and statistical analysis, this invention also includes the use and development of computer systems (machine learning algorithms) that are able to learn and adapt without following explicit instructions, by using algorithms and statistical models to analyze and draw inferences from patterns in data. Furthermore, the invention also included a continuous improvement mechanism whereby the data collected and generated from each additional program participant is included in an expanding database. The expanding dataset (including behavioral health, clinical characteristics, user engagement and other metrics) was used to improve the statistical analysis described above using machine learning.

Using the data provided by the participant and collected in the database, a detailed diet specific to each phase and personalized for each participant can be created in accordance with embodiments of the present invention.

The 37-member cohort who completed the study (16 IBS, 12 UC and 9 CD comprised participants that were 62% female, with an average age was 37, and 73% of participants were white. Primary and secondary outcomes showed that the digital personalized elimination diet program improved symptoms statistically and clinically significantly for a majority of participants. Most participants achieved symptomatic remission and symptom relief persisted for the entire study period.

Statistically significant symptom improvement (p<0.001 Bonferroni-corrected two-tailed t-test) was seen for 81% of participants at week 5 and persisted for 70% of participants at week 9, measured by the relevant symptom severity score. By end of study, participants with IBS (n=16) improved symptoms by an average 59.3 points (IBS-SSS, p<0.001, petasq=0.62), participants with UC (n=12) improved by an average 1.3 points (P-SCCAI, p<0.001, petasq=0.53) and participants with CD (n=9) improved by an average 3.1 points (mHI-CD, p<0.001, petasq=0.72), evaluated by one-way repeated-measures ANOVA.

Symptom improvement at midpoint was maintained until the end of the study for individuals in each condition group. IBS symptoms at study midpoint (117.6+/−60.4 IBS-SSS) and end (104.9+/−63.0) were significantly lower than baseline (170.6+/−59.3, p<0.001), as were UC symptoms (2.9+/−1.3 P-SCCAI at baseline, 1.9+/−1.3 at midpoint and 1.4+/−1.4 at end, p<0.007) and CD symptoms (6.6+/−1.3 mHI-CD at baseline, 3.0 +/−1.9 at midpoint, 3.9+/−1.9 at end, p<0.001), calculated via Bonferroni-corrected post-hoc analysis. No significant difference was found between phase 2 and 4 for any diagnosis group (IBS p=0.66, UC p=0.51, CD p=0.36), indicating symptom relief was maintained successfully 4 weeks after the identification and elimination of personal trigger foods.

Given frequent concomitant IBD and IBS diagnoses, IBS-SSS scores for participants with IBD were also evaluated (total n=37) and showed a 49-point improvement from baseline (p<0.001, petasq=0.53, one-way repeated-measures ANOVA). Symptom relief was similarly maintained until study end (154.3+/−52.8 at baseline, 106.9+/−52.9 at midpoint and 103.8+/−54.9 at end, p<0.001). No significant difference was found between Phase 2 and 4 (p=0.99).

Seventy-eight percent of participants saw clinically significant symptom improvement at week 5 and 62% at week 9 (week 5 vs 9 p=0.18, McNemar's test). Twenty-five participants (67.6%) achieved symptomatic remission by end of study (50%, 62.5% and 77.8% of IBS, UC and CD cohorts, respectively). Participants were 14 times more likely to be in remission at study end compared to baseline (p<0.001, McNemar's test). No significant difference was observed between study midpoint and endpoint (p=0.15), indicating a persistence of clinically significant symptomatic relief. Statistically and clinically significant symptom improvement was observed regardless of sex, age quartile, severity at intake and ethnicity.

The results of the 9-week study are summarized in Table 2, as follows:

| | |
|---|---|
| Baseline | End |
| at intake | (Phase 4) |

All Participants (IBS-SSS) (n = 37)

| | | |
|---|---|---|
| Severe (>300) | 12 (32.2%) | — |
| Moderate (175 < x < 300) | 17 (45.9%) | 6 (16.2%) |
| Mild (75 < x < 175) | 8 (21.6%) | 15 (40.5%) |
| Remission (<75) | — | 16 (43.2%) |

IBS (IBS-SSS) (n = 16)

| | | |
|---|---|---|
| Severe (>300) | 6 (37.5%) | — |
| Moderate (175 < x < 300) | 8 (50.0%) | 3 (18.7%) |
| Mild (75 < x < 175) | 2 (12.5%) | 5 (31.3%) |
| Remission (<75) | — | 8 (50.0%) |

Ulcerative Colitis (p-SCCAI) (n = 12)

| | | |
|---|---|---|
| Severe (12-19) | — | — |
| Moderate (6-11) | 2 (16.7%) | — |
| Mild (3-5) | 10 (83.3%) | 2 (16.7%) |
| Remission (0-2) | — | 10 (83.3%) |

Crohn's Disease (mHI-CD and s-CDAI) (n = 9)
All Participants (IBS-SSS) (n = 37)

| | | |
|---|---|---|
| Active Disease (>5.5 | 9 (100%) | 2 (22.2%) |
| mHI-CD, >150 s-CDAI) | | |
| Inactive Disease (>5.5 | — | 7 (77.8%) |
| mHI-CD, <150 s-CDAI) | | |

Secondary Outcomes: Users reported the diet program was feasible and desirable. Ninety-five percent daily engagement (completed surveys) and 89% adherence with the protocol were observed. Participants reduced their intake of suggested trigger foods by 89.3% on average during the elimination phase (89%+/−13%); adherence was significantly left-skewed (skewness=−1.3, SE=0.39). Higher adherence during trigger food elimination was not associated with increased symptom improvement (p=0.62, 0.70, and 0.76 for IBS, UC and CD, respectively) due to homogeneous adherence, see FIG. 4. Eighty-nine percent of participants (n=33) were fully or partially adherent to the reintroduction protocol. Eighty-five percent of those participants (n=28) were able to re-identify at least one trigger food during reintroduction; these individuals reduced intake of trigger foods in the maintenance phase by an average of 65% relative to baseline. User-reported outcomes related to disease knowledge, quality of life, overall wellbeing and five other factors also demonstrated strong improvement as detailed by symptom improvement reported by each participant as a result of the personalized elimination diet program. Each bar illustrated in the graph represents the percentage of participants who agree with the statement below the bar.

A study using the method and system in accordance with the present invention is described fully in Jactel, Samuel N et al; Efficacy of a Digital Personalized Elimination Diet for the Self-Management of Irritable Bowel Syndrome and Co-Morbid Irritable Bowel Syndrome and Inflammatory Bowel Disease. Clinical and Translational Gastroenterology ( ) 10.14309/ctg.0000000000000545, Nov. 1, 2022. DOI: 10.14309/ctg.0000000000000545;²⁴ the contents of which is incorporated within in its entirety.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention. All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

### REFERENCES CITED

1. Hart, A L et al., What are the top ten research questions in the treatment of inflammatory bowel disease? A priority of setting partnership with the James Linn Alliance. 2017. Journal of Crohn's and Colitis: 204-211.
2. Cohen, BL and DB Sacher, Update on anti-tumour necrosis factor agents and other new drugs for inflammatory bowel disease. 2017. BMJ: 2505.
3. Overview of Crohn's Disease. 2021. The Crohn's and Colitis Foundation, Crohnscolitisfoundation.org/what-is-crohns-disease/overview: 1-5.
4. IBID.
5. IBID
6. IBID
7. Khor B et al. 2011. Genetics and pathogenesis of inflammatory bowel disease. Nature 474:307-317.
8. AxelOrad J E et al. 2016. Inflammatory bowel disease and cancer: the role of inflammation, immunosuppression and cancer treatment. WJG (22): 4794-4801.
9. Hart, A L. Journal of Crohn's and Colitis: 205.
10. Cohen and Sacher, BMJ: 2505.
11. IBID
12. Nemati S and Teimourian S. 2017. An overview of inflammatory bowel disease: general consideration and genetic screening approach in diagnosis of early onset subsets. Midde East J Dig Dis 9:69-80.
13. Cohen and Sacher, BMJ: 2505
14 Axelrad et al. WJG: 4794.
15. Cohen and Sacher, BMJ: 2505.
16. Weaver K R, Melkus G D, Henderson W A. Irritable Bowel Syndrome. Am J Nurs. 2017;117 (6): 48-55. doi: 10.1097/01.NAJ.0000520253.57459.01
17. Vahedi H, Ansari R, Mir-Nasseri M, Jafari E. Irritable bowel syndrome: a review article. Middle East J Dig Dis. 2010; 2 (2): 66-77.
18. Enck, P., Aziz, Q., Barbara, G. et al. Irritable bowel syndrome. Nat Rev Dis Primers 2, 16014 (2016). https://doi.org/10.1038/nrdp.2016.14.
19. McInnes, I. B., Gravallese, E. M. Immune-mediated inflammatory disease therapeutics: past, present and future. Nat Rev Immunol 21, 680-686 (2021). https://doi.org/10.1038/s41577-021-00603-1.
20. Thao Pham, Harry Sokol, Bruno Haliqua et al. Immune-mediated inflammatory diseases and nutrition: results from an online survey on patients' practices and perceptions, 21 Jan. 2021, PREPRINT (Version 1) available at Research Square [https://doi.org/10.21203/rs.3.rs-137887/v1.
21. P. Jacobs et al., Socioeconomic Burden of Immune-Mediated Inflammatory Diseases—Focusing on Work Productivity and Disability. The Journal of Rheumatology Supplement November 2011, 88 55-61; DOI: 10.3899/jrheum. 11090.
22. Jiang Y, Jarr K, Layton C, Gardner CD, Ashouri J F, Abreu M T, Sinha S R. Therapeutic Implications of Diet in Inflammatory Bowel Disease and Related Immune-Mediated Inflammatory Diseases. Nutrients. 2021; 13 (3): 890. https://doi.org/10.3390/nu13030890.
23. Julià, A., Martínez-Mateu, S. H., Domènech, E. et al. Food groups associated with immune-mediated inflammatory diseases: a Mendelian randomization and disease severity study. Eur J Clin Nutr 75, 1368-1382 (2021). https://doi.org/10.1038/s41430-021-00913-6.
24. Jactel, Samuel N et al; Efficacy of a Digital Personalized Elimination Diet for the Self-Management of Irritable Bowel Syndrome and Co-Morbid Irritable Bowel Syndrome and Inflammatory Bowel Disease. Clinical and Translational Gastroenterology ( ) 10.14309/ctg.0000000000000545, Nov. 1, 2022. DOI: 10.14309/ctg.0000000000000545.
25. Loddo and Romano; Inflammatory bowel disease: genetics, epigenetics, and pathogenesis. Front. Immunol. 2015:551. doi: 10.3389/fimmu.2015.00551.
26. Kirsner, Joseph B. World J Gastroentero, 2001:175-184.
27. Mittermaier, C., et al. Impact of Depressive Mood on Relapse in Patients with Inflammatory Bowel Disease: A Prospective 18-Month Follow-Up Study. Psychosomatic Medicine, 2004:79-84.
28. Bitton, A., et al. Psychosocial Determinants of Relapse in Ulcerative Colitis: A Longitudinal Study. The American Journal of Gastroenterology 2003:2203-2208.
29. Mardini, H., et al. Crohn's Disease: A Two-Year Prospective Study of the Association Between Psychological Distress and Disease Activity. Digestive Diseases and Sciences; New York Vol. 49, March 2004:492-7.
30. Schwartz, S. P. and Blanchard, E. B. Evaluation of a Psychological Treatment for Inflammatory Bowel Disease. Behav Res Ther. 1991:167-77.
31. Jantschek et al. Effect of Psychotherapy on the Course of Crohn's Disease. Results of the German Prospective Multicenter Psychotherapy Treatment Study on Crohn's Disease. Scand J Gastroenterol. 1998:1289-96.
32. Milne et al. A Stress Management Programme for Inflammatory Bowel Disease Patients. Journal of Advanced Nursing. September 1986:491-620.
33. Jarrett, Monica E., et al. Comprehensive Self-Management for Irritable Bowel Syndrome: Randomized Trial of In-Person versus Combined In-Person and Telephone Sessions Am J Gastroenterol. 2009:3004-3014.
34. Zia, Jasmine K. et al. A Comprehensive Self-Management Irritable Bowel Syndrome Program Produces Sustainable Changes in Behavior After 1 Year, Clin Gastroenterol Hepatol. 2017:212-219.

What is claimed is:

1. A system for adaptive, multi-level processing of health data from processing levels distributed among at least one computing device comprising a processing unit, a network adapter, an input/output system, and a database wherein
a. the processing unit comprises a central processing unit (CPU) and, optionally, a graphics processing unit (GPU) or multiple processing units, and connects by means of the network adapter to a network, an input and output system, and a database, wherein
b. the network optionally includes a measurement and interaction device;
c. the input and output system receives and collects inputs from the network to transmit to the database and sends outputs from the database to the network; and
d. the database comprises data and at least one computer software program having instructions for extracting, categorizing, reading and analyzing information, generating outputs from the information, and transmitting the outputs to the network.

2. The system according to claim 1, wherein the input and output system receives and sends data to and from the network and wherein the data resides in the database, said data selected from the group consisting of personalized health data, drug and medication data, electronic medical data, behavioral health data, measurement and interaction data; data from wearable devices, therapy data, dietary data, medical testing data, survey data, health plan data, insurance claims data, and combinations thereof.

3. The system according to claim 1, wherein the database further comprises a content module comprising collected and stored content selected from the group consisting of therapeutic exercises, recorded audio programs, recorded video programs, recommended diets, behavioral data, clinical practice guidelines, published research, educational material, peer-to-peer, peer-to-clinician, and self-to-self messages, surveys, and combinations thereof.

4. The database according to claim 3, further comprising one or more software programs or algorithms having instructions for extracting, categorizing, reading and analyzing information, generating outputs, and transmitting those outputs to the network.

5. The database according to claim 4, wherein the algorithms are selected from the group comprising classification algorithms, recommendation algorithms, analysis algorithms, comparison algorithms, and combinations thereof.

6. The database according to claim 4, wherein the algorithms further include machine learning algorithms, artificial intelligence algorithms, and combinations thereof.

7. The system according to claim 1 wherein the processing unit comprises one or more processing devices selected from the group consisting of a microprocessor, a central processing unit, a complex instruction set computing (CISC) microprocessor, a reduced instruction set computing (RISC) microprocessor, a very long instruction word (VLIW) microprocessor, a processor implementing other instruction sets, a processor implementing a combination of instruction sets, an application specific integrated circuit (ASIC), a field programmable gate array (FPGA), a digital signal processor (DSP), a network processor and combinations thereof.

8. The system according to claim 1, wherein the processing unit communicates with the output system configured to periodically communicate with a user of the system through the measurement and interaction device to provide to the user information and recommendations selected from the group consisting of lifestyle modification recommendations, coaching, therapeutic exercises, recorded audio programs, recorded video programs, behavioral data, clinical practice guidelines, reminders, educational material, advice, and combinations thereof.

9. The system according to claim 1, wherein the computing device is connected through the Internet network.

10. The system according to claim 1, in which the at least one computing device and measurement and interaction device is a cell phone, smartphone, tablet, personal computer, or combination thereof.

11. A method for developing a personalized program for the amelioration of signs in a participant having an immune-mediated inflammatory disease comprising:
a. collecting personal information and other data about the participant through an input system and measurement and interaction device through a network;
b. transferring the collected information to a processing unit, said processing unit further comprising a database for data storage and a plurality of algorithms for analyzing the data and wherein the processing unit is in communication with the input system, the database, and the network;
c. analyzing the information collected in the processing unit using a set of algorithms to generate at least one set of personalized results and recommendations relevant to the participant and their immune-mediated inflammatory disease, including but not limited to personalized content, coaching, lifestyle change recommendations, education and the like;
d. storing the results, recommendations and associated data in the database in communication with an output system;
e. communicating information including at least one personalized set of results and recommendations to the participant through an output system and a measurement and interaction device via a network.

12. The method according to claim 10, wherein the information collected from the participant is selected from the group consisting of personalized health data, drug and medication data, electronic medical data, behavioral health data, measurement and interaction data; data from wearable devices, therapy data, dietary data, medical testing data, survey data, health plan data, insurance claims data, and combinations thereof.

13. The method according to claim 10 wherein the personal information and data about a participant is provided by an external data source selected from the group comprising a testing laboratory, a physician's office, a hospital or clinic, a dietitian, a psychotherapist, a mental health counselor, a health and wellness coach, a social worker, a care navigator, a peer, a family member and combinations thereof.

14. The method according to claim 11 wherein the information communicated to the participant comprises at least one personalized recommendation selected from the group consisting of lifestyle modifications, coaching, therapeutic exercises, recorded audio programs, recorded video programs, behavioral data, clinical practice guidelines, reminders, educational material, advice, and combinations thereof.

15. The method according to claim 14 wherein the information is communicated to the participant through the output system and measurement and interaction device.

16. The method according to claim 14 wherein the personalized recommendation consists of at least one program comprising one or more modules selected from the group consisting of stress-reduction techniques, breathing exercises, mindfulness and mediation practice, progressive muscle relaxation techniques, guided imagery and/or visualization techniques, yoga, Tai Chi, biofeedback exercises, music therapy, gut directed hypnosis, acceptance and commitment therapy, education about the mind and gut connection, dietary guidance, sleep improvement techniques, exercise techniques and combinations thereof.

17. The method according to claim 11, wherein the collection of information is iterative such that the collected information is analyzed continually to provide at least one new personalized recommendation based on the continual analysis.

18. The method according to claim 11, wherein the immune-mediated inflammatory disease is selected from the group consisting of inflammatory bowel disease (IBD), irritable bowel syndrome (IBS), Barrett's esophagus, gastroesophageal reflux disease, rheumatoid arthritis, osteoarthritis, spondyloarthritis, connective tissue disorders, psoriasis, atopic dermatitis, asthma, Attention Deficit Hyperactivity Disorder (ADHD), Type 1 diabetes, multiple sclerosis and combinations thereof.

19. The method according to claim 18, wherein the inflammatory bowel disease is selected from the group consisting of Crohn's disease, ileocolitis, ileitis, gastroduodenal Crohn's Disease, jejunoileitis, Crohn's colitis and ulcerative colitis.
